(19) United States
(12) Patent Application Publication
(10) Pub. No.: US 2023/0138173 A1
(43) Pub. Date: May 4, 2023

(54) MARKING MODULE FOR AN APPLIANCE INTENDED FOR APPLYING A COLORANT, AND APPLIANCE USING SUCH A MARKING MODULE

(71) Applicant: ARMINK TATTOO BODY ART, JOUGNE (FR)

(72) Inventors: Steven KAUFFMANN, Yverdon-les-Bains VD (CH); Vincent REVOL, Rothenburg LU (CH); Tormod VOLDEN, Luzern LU (CH)

(73) Assignee: ARMINK TATTOO BODY ART, JOUGNE (FR)

(21) Appl. No.: 17/913,255

(22) PCT Filed: Apr. 8, 2021
(86) PCT No.: PCT/EP2021/059236

§ 371 Date: Sep. 21, 2022

(30) Foreign Application Priority Data
Apr. 9, 2020 (CH) 00435/20

(51) Int. Cl. A45D 34/04; A61M 37/00

(52) U.S. Cl. CPC A45D 34/04; A61M 37/0084

(57) ABSTRACT

A marking module for an appliance intended for applying a colorant for permanent makeup or tattooing has a channel and at least one needle that is able to move longitudinally in said channel between a retracted position in which a distal end of the needle is located inside said channel and an extended position in which the distal end of the needle protrudes from said channel. The marking module has a reservoir of colorant and said reservoir communicates with said channel only in a region situated downstream of the needle when this needle is located in said retracted position. An appliance for applying a colorant for permanent makeup or tattooing has a marking module as defined above and a module for actuating the needle of the marking module.

### TECHNICAL FIELD

The present invention relates to the field of permanent makeup or tattooing. More specifically, the invention relates to a marking module for an apparatus intended for applying a colorant in order to make a permanent makeup or a tattoo, the marking module comprising a channel and at least one needle movable longitudinally in said channel between a retracted position in which a distal end of the needle is within said channel and an extended position in which the distal end of the needle protrudes from said channel.

The invention also relates to an apparatus for applying a colorant for making permanent makeup or for tattooing.

### PRIOR ART

Currently, apparatuses intended for applying a colorant to a person and for making permanent makeup or tattooing are consisting of a gun in which a tattoo needle moves longitudinally, and a drive intended for moving the needle. When a colorant such as in particular an ink must be applied onto or under the skin of a user, the needle is dipped in an ink reservoir and then applied to the skin of the user such that the ink penetrates the user's skin. When the ink picked up by the needle is consumed, the needle is dipped again in the ink reservoir, and then the cycle starts again.

One problem encountered with this type of apparatuses is that the amount of ink that can be used between each dip in the reservoir is relatively small. When the person making the tattoo needs, for example, to draw a straight or elongated line, he must regularly stop drawing to pick up ink. Resume drawing right where the person left off is relatively difficult and therefore the drawing may not be perfect.

In addition, the time required to make a tattoo can be long due to ink refilling that is necessary to carry out regularly. This is all the more important especially when large surfaces have to be coloured, for example when shading. This relatively long working time may be unpleasant for the person making the tattoo or makeup, but especially for the person being tattooed or on whom the makeup is applied. Indeed, under-skin marking can be relatively painful. A shorter working time is therefore more comfortable to the person on whom the marking is performed.

Another problem encountered is that the reservoirs containing the ink are open to allow ink pickup. The ink is therefore in contact with air. Therefore it may be contaminated by pathogens which may then be injected under the skin of the person being tattooed or on whom the makeup is applied, which can cause infections.

To address some of these disadvantages, a tattoo or permanent make-up apparatus comprising a reservoir integrated into the gun exists. Such an apparatus is described in U.S. Pat. No. 6,345,553. This document describes a tattoo or permanent makeup gun comprising a module in which a tattoo needle moves longitudinally driven by a drive. The tattoo gun includes an ink reservoir. During its alternating longitudinal movement, the needle moves in the ink reservoir, then its free end protrudes from the tattoo gun so as to apply the ink picked up by the needle, under the skin of the user.

This implementation largely avoids refilling ink in an external reservoir. It is therefore possible to draw relatively long lines and fill large areas without having to refill ink. In addition, the ink is not exposed to open air and the risks of contamination or infections are therefore minimized. However, this gun has other drawbacks. In fact, ink is introduced into the reservoir and then the needle moves in the reservoir. It is not possible to empty the ink from the reservoir and replace it with a different colour ink. In fact, this would lead to a heterogeneous mixture of colours that could not be used. Thus, if the ink contained in the reservoir is not completely consumed, it goes to waste.

Furthermore, the ink reservoir in which the needle moves is in a position relatively far from the free end of the needle, i.e. from the end from which the needle emerges to make the tattoo. In order for ink to be picked up by the needle, its end must enter entirely into the reservoir. This makes it necessary to carry out a particularly rigid guidance of the needle, which complicates making of the gun from the mechanical point of view and makes it more expensive. In addition, due to the construction of the gun, it is necessary that ink be available on the side of the reservoir where the needle emerges. This forces the person performing the tattoo to hold the gun in a certain way, with the needle substantially vertical pointing downwards, otherwise the needle will not receive ink. This makes this gun inconvenient to use.

Another disadvantage of this device is that the needle is immersed in the ink. This implies that the only ink that can be used is that contained in the reservoir. Therefore, it is not possible to use the ink of the reservoir for a portion of a tattoo and then use the device by dipping the needle in ink from an external reservoir to make another portion of the tattoo.

As a result, in practice the solution described in U.S. Pat. No. 6,345,553 is not ideal. In fact, it is not possible to change ink colour while keeping the same gun. Unused ink is wasted and the gun must be handled in a certain way otherwise it will not operate. Furthermore, it is not possible to use the device alternately in a “conventional” mode in which the needle is dipped in an external ink reservoir, and in the mode using the ink contained in the reservoir integrated into the device.

There is therefore a need for a tattoo or permanent makeup apparatus which is flexible and convenient to use, i.e. which in particular allows continuous use without having to regularly dip the needle in ink, enables changing the ink colour without wastage, which can be used in normal positions that a person performing a tattoo conventionally adopts, and which can be used in two different modes.

### DESCRIPTION OF THE INVENTION

Such an apparatus is described in this document.

The objects of the invention are achieved by a marking module as defined in the preamble and characterised in that it includes a colorant reservoir and in that said reservoir communicates with said channel only in an area located downstream of the needle when the needle is in said retracted position.

The objects of the invention are also achieved by an apparatus for applying a colorant as defined in the preamble and characterised in that it comprises a marking module as defined above, and an actuation module of the needle of the marking module.

The marking module according to the present invention can be used in all kinds of positions, which makes it possible to make a permanent make-up or a tattoo without either the person performing the tattoo or the person being tattooed having to be placed in an uncomfortable position.

The tattoo can be performed in a shorter time than with a conventional apparatus, especially if the design is large and/or has large areas to fill. Reducing the duration is comfortable both for the person who performs the marking and for the person on whom the marking is performed.

In addition, with the device of the invention, it is possible to add ink to a reservoir in a simple and practical way, the amount of ink added corresponding to the needs and not to the capacity of the reservoir.

It is also possible to change the colour of the ink in a simple way, which allows the use of the same marking module for several colours. Furthermore, it is possible to use pre-filled cartridges, which avoids manipulation of the ink. It is also possible to prevent the ink from remaining in the open air for a long time thereby avoiding the risk of contamination by pathogens contained in the air.

The apparatus of the invention enables the use of the ink contained in the cartridge or in the reservoir integrated into the device. It also allows the reservoir to be closed and the device to be used conventionally, i.e. by dipping the needle in ink contained in an external reservoir.

This is particularly interesting in the case where a significant portion of a tattoo or makeup is made using one colour while another portion requires the use of another colour or in the case where two colours must be used alternately. In such a case, it is not necessary to empty the integrated reservoir, clean it and refill it with another colour to make a portion of the tattoo or makeup. One has to simply close the reservoir, and then use the device in the conventional way, by picking up ink with the needle from an external reservoir.

When this portion of the tattoo or make-up is finished, the reservoir can be reopened and the ink from the integrated reservoir is used again.

### BRIEF DESCRIPTION OF THE DRAWINGS

The present invention and advantages thereof will be better understood with reference to the appended figures and the detailed description of a particular embodiment, in which:

FIG. 1 illustrates a marking module according to a first embodiment of the present invention;

FIG. 2 is a cross-sectional view of a portion of the marking module of FIG. 1, in a first configuration;

FIG. 3 is a view similar to FIG. 2, in a second configuration, with the needle in a retracted position;

FIG. 4 is a view similar to FIG. 3, with the needle in forward position;

FIG. 5 is a view similar to FIG. 3, with the needle in an extended position;

FIG. 6 is a perspective view of the portion of the marking module of FIG. 2;

FIG. 7 illustrates a marking module according to a second embodiment of the invention, in which an ink reservoir is separated from the body of the marking module;

FIG. 8 represents the marking module of FIG. 7, with the ink reservoir partially placed on the body of the marking module;

FIG. 9 represents the marking module of FIG. 7, with the ink reservoir placed on the body of the marking module;

FIG. 10 is a partial cross-sectional view of the marking module of FIG. 7, with ink contained in the reservoir;

FIG. 11 is a partial cross-sectional view of a removable cartridge, in a first configuration;

FIG. 12 is a view similar to FIG. 11, in a second configuration;

FIG. 13 illustrates an ink reservoir used in a marking module according to the present invention as well as a cartridge for adding ink to the reservoir; and

FIG. 14 illustrates a marking module according to the present invention, being cleaned

### EMBODIMENT OF THE INVENTION

With reference to the figures, the invention relates to an apparatus 10 for applying a colorant, used in particular for making permanent make-up or for tattooing. This apparatus is essentially made up of two parts, i.e. a marking module 11 comprising in particular a needle 12, and an actuation module 110 of the needle. According to a preferred embodiment, the marking module 11 is removable such that a marking module can be mounted on an actuation module, detached therefrom and replaced by another marking module. In practice, the actuation module 110 includes at least one drive and is used to perform a large number of tattoos whereas several marking modules can generally be used for one person. Due to hygiene reasons, a marking module is generally single-use, i.e. it is used to tattoo only one person and is disposed of after use on that person.

The actuation module 110 may be of any conventional type and is therefore neither described nor shown in detail in the figures. It is simply provided to removably receive a marking module.

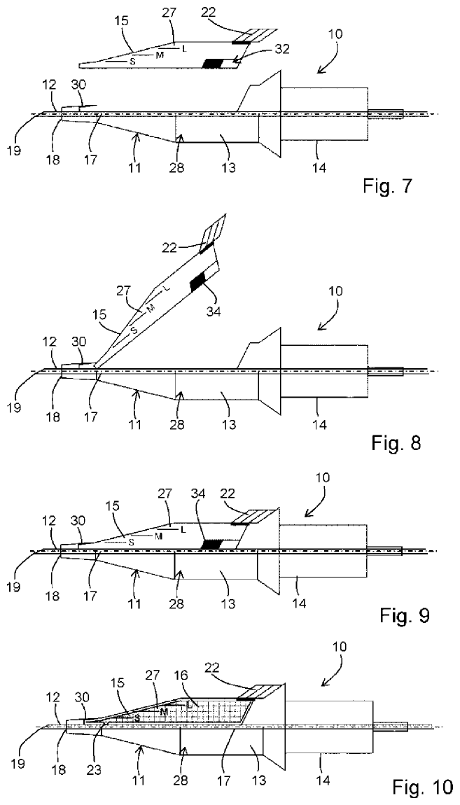

The marking module 11 includes a body 28 provided with a handle 13 arranged to be easily held by the user, and a sleeve 14 intended for introduction into the actuation module 110. This marking module 11 further includes the needle 12 mentioned above and a colorant reservoir 15 such as in particular ink 16. The reservoir may be stationary and form an integral part of the marking module or include a removable cartridge 27 as shown in FIGS. 7 to 14, described in detail below.

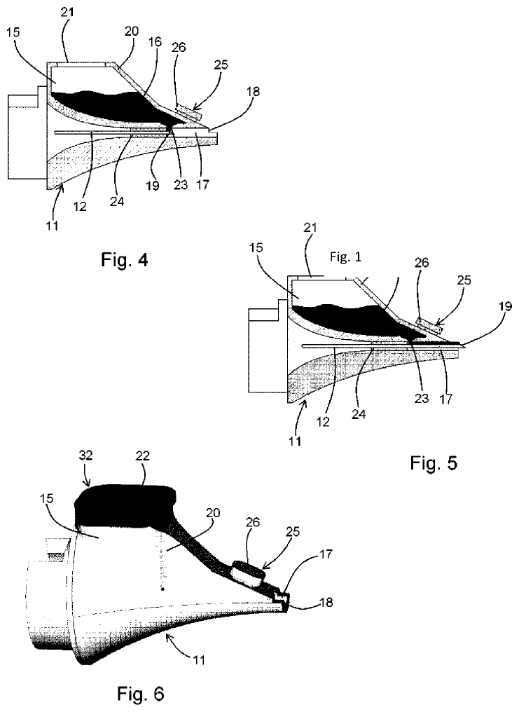

As can be seen in particular in FIGS. 2 to 6, the marking module 11 includes a longitudinal channel 17 in which the needle 12 undergoes a reciprocating movement, under the effect of the actuation module. The channel 17 opens into one end of the marking module, called free end 18, from which the needle 12 can protrude from the channel and come into contact with the skin of the person to be tattooed so as to deposit ink thereon. The needle reciprocates between a retracted position shown in FIG. 2, in which a distal end 19 of the needle 12 is fully within the channel 17, and an extended position shown in FIG. 5, in which the distal end 19 of the needle 12 protrudes from the channel 17 and can come into contact with the skin of the person on whom the marking is performed.

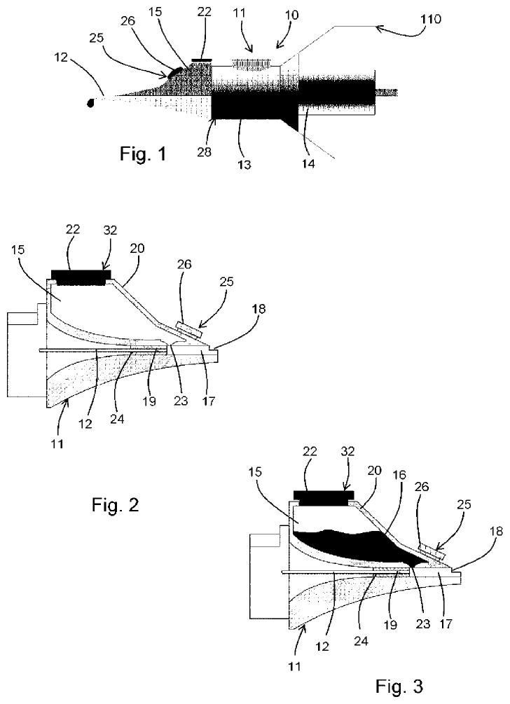

In the embodiment illustrated by FIGS. 1 to 6, the ink reservoir 15 includes essentially a sealed wall 20, a filling opening 21 closed by a cap 22, and a lumen 23 disposed near the free end 18 of the channel 17 and connecting the inside of reservoir 15 to the channel 17.

In the embodiment illustrated by FIGS. 7 to 14, the ink used for marking or tattooing is placed in the removable cartridge 27 described in detail below and acting as a reservoir 15.

When the marking module 11 is used according to a normal operating mode, the needle 12 moves longitudinally in the channel 17, under the effect of the actuation module 110. In its fully retracted position, i.e. in the position in which the distal end 19 of the needle 12 is farthest from the free end 18 of the channel, the tip of the needle 12 is upstream of lumen 23 of the reservoir 15. The lumen is dimensioned so as to allow the passage of a small amount of ink 16 in the form of a droplet. When the needle 12 is advanced in the direction of the free end 18 of the channel, it passes close to the lumen 23 and it carries with it the droplet of ink 16 which it can then deposit under the tattooed person's skin, conventionally. At each cycle of retraction of the needle 12 in the channel 17 and displacement of the needle forwards, at the free end 18 of the channel 17, the needle 12 picks up a droplet of ink 16 from the reservoir 15.

The marking module 11 comprises a guide 24 disposed in the channel 17. The guide 24 extends advantageously over an area between the location where the lumen 23 opens into the channel 17 and an area upstream of the location of the tip or the distal end 19 of the needle 12 when this needle is in its fully retracted position. This has the advantage that the needle 12 has always a portion contained in the guide 24 and is therefore always adequately guided. In addition, the guide 24 prevents ink 16 from being drawn upstream of the lumen 23 when the needle 12 moves from the extended position to the retracted position. The guide 24 may be disposed for example only on the lumen 23 side or may be a pierced cylinder placed in the channel, all around the needle.

According to a first embodiment of the reservoir 15, the latter includes a priming member 25, the function thereof is to push a first drop of ink 16 into the lumen 23. This priming member 25 may for example be formed of a flexible area in the sealed wall of the reservoir 15, this flexible area can be deformed for example using a button 26. Pressing the button 26 generates an overpressure inside the reservoir, which pushes ink towards the open area of the reservoir, i.e. the lumen 23. When a first drop of ink 16 has been formed in the lumen 23, other drops are formed by capillarity when the first droplet is picked up by the needle 12.

According to a second embodiment of the reservoir 15, the entire reservoir 15 may be made of a flexible material, so that the priming is carried out by pressing any portion of the reservoir.

According to a third embodiment, the reservoir 15 comprises a pressurized air inlet, generating an overpressure inside the reservoir. The pressurized air may, for example, be generated by the actuation module, a portion of which acts as a compressor. In this embodiment, it is possible to provide a member (not shown) for adjusting the pressure generated in the reservoir, the adjustment of the pressure allowing the adjustment of the ink flow from the reservoir into the channel through the lumen. Such pressure and/or flow adjustment is useful, for example, for adapting the amount of ink delivered according to the type of line to make.

According to an alternative, it is also possible to adjust the flow of the ink 16 by adjusting the size of the lumen 23. For this purpose, it is possible to produce a lumen 23 having an area provided with a flexible and deformable wall, the deformable wall being in contact with an adjustment screw (not shown) accessible from outside the reservoir or the marking module 11. In the absence of constraint, the deformable wall of the lumen is in a position such that it allows the passage of a maximum amount of ink. When the adjustment screw is used, it constrains the deformable wall such that the cross-sectional area of the lumen is reduced and the lumen allows the passage of a smaller amount of ink.

According to an alternative, it is possible to replace the pressurized air inlet in the reservoir 15 by a suction or vacuum carried out at the channel. The suction can be achieved using a vacuum pump (not shown) connected to the channel 17, proximate to the free end 18 thereof. As for the compressor, the vacuum pump may be obtained from the actuation module. Ink flow may also be adjusted by a flow adjuster.

When ink must be added to the reservoir 15, for example when the reservoir is empty, the ink can simply be added through the opening 21 of the reservoir either by removing the cap 22 and pouring the ink 16 in the reservoir 15, or through a lid of the cap 22, which can be interesting to ensure the sterility of the ink. Advantageously, the cap 22 of the reservoir is not hermetically closed during use of the tattoo apparatus, which allows passage of air into the reservoir as the ink is consumed.

According to an alternative, the marking module of the invention includes a retention mechanism 32 for retaining the ink 16 in the reservoir 15. The retention mechanism 32 is provided to prevent the ink from exiting the reservoir to the channel 17 when the retention mechanism is activated.

If the retention mechanism 32 is activated, the ink in the reservoir is not used. It is instead possible to use ink from an external reservoir, which can be interesting, for example, when a small portion of the tattoo or marking must be made with an ink of a different colour. When that portion of the tattoo or marking is complete, the retention mechanism 32 can be deactivated and the ink in the reservoir can be used again. The retention mechanism is also useful for transporting and storing cartridges, to prevent ink from spilling out unintentionally.

According to a particular embodiment, the cap 22 of the reservoir can adopt three distinct positions. In a first position, it is open and allows the cartridge 27 to be filled. In a second position, the cap 22 is hermetically closed. In this position, air cannot enter the cartridge 27 or reservoir 15. The ink cannot flow out through the lumen 23 and the reservoir 15 is completely closed. This position is represented by FIG. 2. The cap acts as a retention mechanism 32. The hermetically closed position of the cap corresponds to the activated position of the retention mechanism.

In a third position, the cap 22 is non-hermetically closed. This allows the passage of air into the reservoir 15, which allows the ink to flow towards the lumen 23. This position is represented by FIG. 3. It corresponds to the deactivated position of the retention mechanism 32.

When the cap 22 is placed in its non-hermetically closed position, the marking module 11 can be used as previously described. Ink 16 from the reservoir 15 passes through the lumen 23 and is picked up by the needle 12.

According to an alternative of the retention mechanism, the stroke of the needle 12 can be adjusted by the actuation module 110. In a first operating mode of the marking module, the stroke of the needle is between a first position in which the distal end 19 protrudes from the channel 17 of the marking module and extends beyond the free end 18 of the channel 17, and a second position in which the distal end 19 of the needle is upstream of the lumen 23, as illustrated in FIG. 3.

In a second operating mode, the stroke of the needle 12 is between a first position identical to the first position described above and a second position in which the distal end 19 of the needle 12 is downstream of the lumen 23 and an area of the needle 12 is always opposite the lumen 23.

In this case, the lumen 23 is always closed and the ink in the reservoir cannot flow. Ink from an external reservoir can then be used. In this case, the stroke of the needle 12 acts as a retention mechanism 32 of the ink. When the stroke of the needle brings the distal end 19 thereof upstream of the lumen 23, the retention mechanism 32 is deactivated. When the distal end 19 of the needle is still downstream of the lumen 23, the retention mechanism 32 is activated in that the needle prevents ink from flowing through the lumen.

Instead of blocking the lumen 23 by means of the needle 12 when the retention mechanism 32 is activated, the lumen 23 may be blocked by the guide 24. The latter could in fact be movable between a forward position in which it blocks the lumen 23 and a rearward position in which it releases the passage of ink into the lumen.

FIGS. 7 to 14 describe an alternative of the marking module according to the present invention. In this alternative, the ink 16 used for marking or tattooing is contained in a reservoir 15 formed by the cartridge 27 which is separable from the body 28 of the marking module 11. FIG. 7 illustrates the marking module in which the ink cartridge 27 is separated from the body 28.

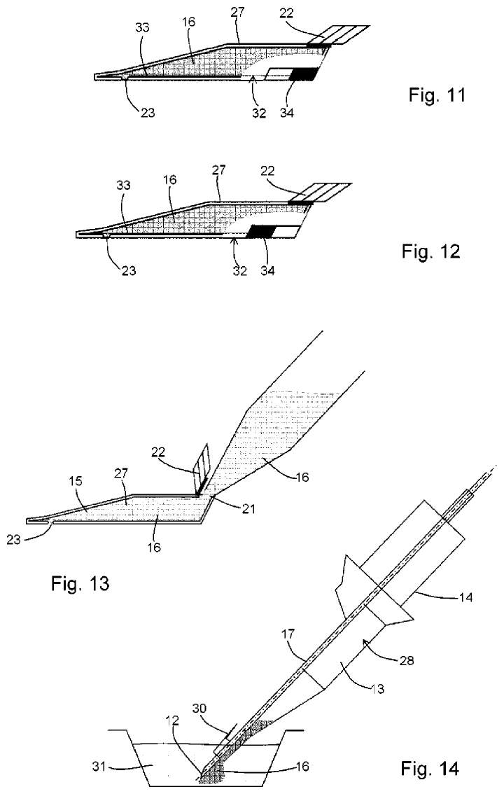

As can be seen in particular in FIGS. 11 and 12, the cartridge 27 includes a lumen 23 allowing the ink to pass from the cartridge to the channel 17 of the body or directly in contact with the needle 12.

The cartridge 27 further includes a cap 22 which is at least operable to open and/or close the reservoir 15, in particular to fill the cartridge.

As described above, the cap 22 can adopt three distinct positions, i.e. an open position, a hermetically closed position and a non-hermetically closed position. However, it could also have only two positions, i.e. an open position and a non-hermetically closed position.

The cartridge 27 preferably includes a locking mechanism (not shown) for locking the cartridge in position when it is placed on the body 28 of the marking module. When the locking mechanism is in the unlocked position, it is possible to remove the cartridge 27 from the body 28 of the marking module. When the locking mechanism is in the locked position, the cartridge 27 cannot be removed from the body of the marking module.

The cartridge 27 may further include graduations and may be partially transparent so as to allow the amount of ink contained in the reservoir to be visible and to be estimated.

When a cartridge 27 must be placed on the body 28 of a marking module 11, the locking mechanism is positioned in an open or unlocked position. An area of the cartridge 27 is placed in a retaining area 30 of the body of the marking module, as shown in FIG. 8 and then the cartridge 27 is placed in its final position, as shown in FIG. 9. The locking mechanism is then actuated so as to lock the cartridge 27 in its operating position. When the cartridge 27 is in place, the lumen 23 is placed proximate to the needle 12. In its rearward position, the distal end 19 of the needle 12 is upstream of the lumen 23. When moving forward, the needle picks up a droplet of ink which it can then deposit under the skin of the person to be tattooed.

When the ink cartridge 27 must be refilled, the cap 22 is opened and ink is introduced through the opening of the reservoir 15. The amount of ink introduced can be selected so as to correspond substantially to the required amount of ink for the selected colour, which avoids wastage.

The retention mechanism 32 is integrated into the cartridge 27. The retention mechanism 32 is illustrated in more detail by FIGS. 11 and 12 and includes a tab 33 disposed inside the cartridge 27 and a slider 34 accessible from outside the cartridge and integral with the tab 33. The tab is disposed such that it can move between two positions, i.e. an open position in which the lumen 23 is completely unobstructed and the tab 33 does not interfere with the lumen, and a closed position in which the tab 33 blocks the lumen 23 and prevents ink from exiting the reservoir through the lumen. The open position is illustrated by FIG. 11 and the closed position is illustrated by FIG. 12.

When the tab 33 is in the open position or in other words, when the retention mechanism 32 is deactivated, ink 16 can exit the reservoir 15 through the lumen 23 and the marking module 11 can be used by picking up the ink from the reservoir 15.

When the tab 33 is in the closed position or when the retention mechanism 32 is activated, the ink cannot exit the reservoir 15 through the lumen 23. The marking module 11 can then be used in the same way as the conventional marking modules, i.e. by picking up ink from an external reservoir.

In the case of a reservoir 15 made as a removable cartridge 27, it is possible to connect the locking mechanism to the ink retention mechanism 32, such that the retention mechanism 32 is always activated if the locking mechanism is not in the locked position. This prevents ink from leaking out of the reservoir until the cartridge is locked in position on the body of the marking module.

It should be noted that a cartridge 27 can be placed on different marking modules 11, more specifically marking modules with needles of different shapes. For example, the same cartridge can first be placed on a marking module having a specific needle for making fine lines, and then the same cartridge can be placed on a marking module having a specific needle for filling large surfaces and/or for shading.

The use of cartridges also allows the placement of pre-filled cartridges 27, without the need for these cartridges to be filled by the user himself.

Furthermore, given that the ink picked up by the needle 12 passing in front of the lumen 23 is deposited under the user's skin, there is no element transmitted “counter flow”, from the user's skin to the ink inside the cartridge. As a result, the same ink cartridge could be used to tattoo several people without there being a risk of pathogen transmission from one person to another.

In particular, when changing the colour, there may be a very small amount of ink on the end of the needle 12. The end of the marking module at which the distal end of the needle is located may be placed in a container 31 containing a cleaning liquid, as illustrated by FIG. 14. Any remaining ink can then be removed and a new ink cartridge can be put in place, for example with an ink of different colour. However, there is no risk for inks of different colours to mix, since only a very small amount of ink is in contact with the needle.

Some characteristics have been described in relation to the embodiments of FIGS. 1 to 6 and other characteristics have been described in relation to the embodiments of FIGS. 7 to 14. These characteristics are for example those pertaining to the priming of the ink into the lumen 23, the non-hermetically closed position of the cap 22 or the ink retention mechanism 32. It should however be noted that these characteristics can also be achieved with the other embodiment, for which they are not specifically described.

Unlike other tattoo apparatuses with an ink reservoir, there is no need to completely fill the reservoir. It is possible to add or introduce into the reservoir, only the necessary amount of ink.

If an ink of different colour must be used, according to a first alternative, the ink of the first colour is taken out of the reservoir 15 simply by opening the cap 22 and emptying the reservoir. The latter can possibly be cleaned if necessary, to remove any remaining ink on the wall of the reservoir. Since the needle 12 picks up the ink drop by drop, it is not completely covered with ink and therefore changing the colour does not require cleaning of the needle.

According to a second alternative, the ink 16 can be contained in a removable cartridge. In this case, the cartridge may include a closure element which can be opened to allow the passage of ink from the removable cartridge to the channel. This closure element may for example include a lid which is removed by the user when the removable cartridge is put in place, or which is pierced by a tip disposed proximate to the lumen 23. The closure element can also be formed by the retention mechanism 32. The latter may comprise a mechanical element which is moved when the cartridge is put in place, so as to allow the ink to flow from the cartridge to the channel 17, and which blocks the passage between the cartridge and the channel 17 when the cartridge is removed from the reservoir. Such a mechanical element could be a ball or a flap for example. In this way, since the needle 12 is not in the ink, it is easy to change the colour of ink without changing the needle.

The retention mechanism 32 can be activated to prevent the use of the ink contained in the cartridge 27, so as to allow the use of ink contained, for example, in an external reservoir or so as to allow the storage and transport of the cartridge without the risk of ink spillage.

The passage of ink can also be adjusted by means of a cap 22. When the cap is placed in its hermetically closed position, air cannot enter the reservoir and the ink cannot flow, due to the small diameter of the lumen 23. When the cap 22 is in its non-hermetically closed position, air can enter the reservoir and ink can flow through the lumen.

The present invention allows the use of an apparatus for tattooing or for making permanent makeup without having to regularly stop to add ink to the tattoo needle. It allows easy and quick marking of large areas and colour changing in a simple way. It can also be used in two different modes, i.e. one mode in which ink from the reservoir 15 is used, and another mode in which ink from an external reservoir is used. Mode selection can be done quickly and easily, by activating or deactivating the ink retention mechanism. The retention mechanism can of course also be used to prevent ink from leaking, particularly when the marking module is not used and/or when transporting or storing removable ink cartridges.

Thanks to these different characteristics, the apparatus is easier and more pleasant to use for the person making the makeup or tattoo. The same operations can be carried out in a much shorter time than with the apparatuses of the prior art, which is advantageous for the person making the tattoo, but especially for the person being tattooed. In fact, since a tattoo can be relatively painful, saving time when making a tattoo implies greater comfort for the person being tattooed.

The present invention has been described with a dye produced in the form of a liquid ink. It is also possible to use a colorant produced, for example, in the form of a powder or a paste, provided that this powder can be picked up by the needle during its alternating movement inside the channel. This can be particularly achieved by generating an overpressure in the reservoir or a vacuum in the channel, so as to suck the powder or paste into this channel.

1. A marking module for an apparatus for applying a colorant for making permanent make-up or for tattooing, the marking module including:
a channel; and
at least one needle movable longitudinally in said channel between a retracted position in which a distal end of the needle is inside said channel and an extended position in which the distal end of the needle protrudes from said channel,
wherein the marking module includes a reservoir of colorant and wherein said reservoir communicates with said channel only in an area located downstream of the distal end of the needle when the needle is in said retracted position.

2. The marking module according to claim 1, wherein the reservoir includes a lumen opening into said channel in which the needle moves and wherein the reservoir communicates with said channel via the lumen.

3. The marking module according to claim 1, wherein the reservoir includes a removable cartridge.

4. The marking module according to claim 3, comprising a body, wherein the marking module includes a locking mechanism able to adopt a locking position in which the removable cartridge is held in position on said body, and an unlocked position in which the removable cartridge can be separated from the body.

5. The marking module according to claim 1, further comprising a retention mechanism for retaining the ink in said reservoir, the retention mechanism being arranged to prevent the ink from exiting the reservoir to the channel when said retention mechanism is activated.

6. The marking module according to claim 1, wherein the reservoir includes a cap capable of adopting a hermetically closed position in which air cannot enter the reservoir, a non-hermetically closed position in which air can enter the reservoir without ink being able to be introduced into the reservoir and an open position in which air and ink can enter and exit the reservoir.

7. The marking module according to claim 6, wherein said cap is part of the retention mechanism, the retention mechanism being activated when the cap is in its hermetically closed position.

8. The marking module according to claim 5, wherein said needle is part of the retention mechanism, the retention mechanism being activated when the displacement of the needle in the direction of its retracted position is limited to a position in which the distal end of said needle is always downstream of the lumen.

9. The marking module according to claim 5, wherein the retention mechanism includes a tab movable between a closed position in which the tab blocks the lumen and an open position in which the lumen is open.

10. The marking module according to claim 1, wherein the reservoir includes a priming member for priming the arrival of colorant in said channel.

11. The marking module according to claim 1, further comprising a device for generating pressure inside the reservoir.

12. The marking module according to claim 1, further comprising a device for generating vacuum inside the channel.

13. The marking module according to claim 1, further comprising a member for adjusting the flow of ink from the reservoir into the channel.

14. The marking module according to claim 1, further comprising a member for guiding the needle, disposed upstream of said lumen.

15. An apparatus for applying a colorant for making permanent make-up or for tattooing, the apparatus comprising:
the marking module according to claim 1; and
an actuation module of the needle of the marking module.

16. The marking module according to claim 2, further comprising a member for adjusting the flow of ink from the reservoir into the channel.

17. The marking module according to claim 3, further comprising a member for adjusting the flow of ink from the reservoir into the channel.

18. The marking module according to claim 4, further comprising a member for adjusting the flow of ink from the reservoir into the channel.

19. The marking module according to claim 5, further comprising a member for adjusting the flow of ink from the reservoir into the channel.

20. The marking module according to claim 6, further comprising a member for adjusting the flow of ink from the reservoir into the channel.
